(19) United States
(12) Patent Application Publication
(10) Pub. No.: US 2024/0131316 A1
(43) Pub. Date: Apr. 25, 2024

(54) Compliant Port to Catheter Adapter

(71) Applicant: Bard Peripheral Vascular, Inc., Franklin Lakes, NJ (US)

(72) Inventor: Bradley Forsyth, Sandy, UT (US)

(21) Appl. No.: 18/277,958

(22) PCT Filed: Feb. 17, 2022
(86) PCT No.: PCT/US22/16853

§ 371 Date: Aug. 17, 2023

Related U.S. Application Data
(60) Provisional application No. 63/151,525, filed on Feb. 19, 2021.

(51) Int. Cl. A61M 39/10

(52) U.S. Cl. CPC A61M 39/1011; A61M 39/105

(57) ABSTRACT

Embodiments disclosed herein are directed to a coupling device including an invertible sleeve transitionable between a first, unlocked configuration and a second, locked configuration. A stem of the coupling device can be urged into a lumen of the catheter. A stem of a port can be urged into a lumen of the coupling device. The sleeve can be inverted from the unlocked configuration, where the sleeve extends distally, to a locked configuration where the sleeve extends proximally. The sleeve can fit tightly about a sleeve bearing portion of the port in the locked configuration to secure the port thereto. The sleeve can be formed of an elastic material to allow the sleeve to transition between the first stable configuration and the second stable configuration. Advantageously, the coupling device forms a continuous outer profile to mitigate tissue ingrowth.

### PRIORITY

This application claims the benefit of priority to U.S. Provisional Application No. 63/151,525, filed Feb. 19, 2021, which is incorporated by reference in its entirety into this application.

### SUMMARY

Briefly summarized, embodiments disclosed herein are directed to a compliant, port to catheter coupling device, or “cathlock.” The coupling device can include a sleeve that is invertible, or “bi-stable,” in both a first configuration and a second configuration. The coupling device can be configured for securing a subcutaneous port, or similar medical device, to a catheter. Advantageously, the coupling device can facilitate exchange of one of the port or the catheter while providing a secure connection therebetween, and mitigate ingrowth into the components of the port/catheter assembly.

Proximally trimmable catheters allow for post-placement sizing of the catheter. When placing a catheter and port assembly subcutaneously, the position of the distal tip of the catheter can be important for the efficacy of the treatment. For example, when placing a catheter tip within the superior vena cava, if the distal tip of the catheter falls short of the target area, the efficacy of the medicament is reduced. If the distal tip is advanced too far, the distal tip can cause arrhythmia. The distance between the distal tip of the catheter and the port can vary since the distances between the target location, insertion site to the vasculature, and the location of the port can vary between patients and procedures. Estimating the catheter length before placement can lead to errors that result in misplacement of the catheter distal tip.

Proximally trimmable catheters allow for placement of the catheter distal tip at the target location before trimming a proximal portion of the catheter to the correct length. The clinician can then attach the catheter to a subcutaneous port, or similar access device. However, securing the catheter to the port can be challenging. The connection must be leak-proof, especially under high-pressure infusion. Further, manipulating the catheter and port within the confined, wetted environment of a subcutaneous access site can lead to slippage, undue trauma to the access site, or misplacement of the catheter distal tip.

Embodiments disclosed herein are directed to bi-stable, invertible coupling device systems and associated methods, which facilitates exchange of one or both of the port and catheter while mitigating tissue ingrowth into components of the port-catheter assembly.

Disclosed herein is a coupling device for releasably securing a catheter to a port including, a stem receiving portion configured to receive a stem of the port, the stem receiving portion including a lumen, and an invertible sleeve coupled to an outside surface of the stem receiving portion, the invertible sleeve having a first configuration extending toward a distal end of the stem receiving portion, and a second configuration extending away from the distal end of the stem receiving portion.

In some embodiments, a rim portion of the invertible sleeve in the first configuration extends distally from the stem receiving portion to surround a portion of the stem receiving portion.

In some embodiments, the invertible sleeve in the second configuration extends proximally to define a recess configured to receive a portion of the port therein.

In some embodiments, the portion of the port is a sleeve bearing portion.

In some embodiments, a diameter of the recess is the same as, or less than, a diameter of the sleeve bearing portion of the port.

In some embodiments, an inner profile of the recess mirrors and outer profile of the sleeve bearing portion of the port.

In some embodiments, an inner surface of the recess includes a ridge configured to engage a suture groove disposed on the port, when the invertible sleeve is in the second configuration.

In some embodiments, an outer surface of the invertible sleeve in the second configuration and an outer surface of a body of the port forms a continuous outer profile.

In some embodiments, the sleeve is formed of an elastic material selected from a group consisting of a plastic, polymer, elastomer, silicone rubber, synthetic rubber, natural rubber, latex, or composite.

In some embodiments, the coupling device further includes a stem extending distally from the stem receiving portion and configured to engage a lumen of a catheter.

In some embodiments, an outer diameter of the stem of the coupling device is the same or larger than an inner diameter of a lumen of a catheter to engage the catheter in a friction fit.

In some embodiments, an outer diameter of the stem of the port is the same or slightly larger than an inner diameter of the lumen of the stem receiving portion.

In some embodiments, the sleeve in the second configuration is configured to create an air tight seal between a surface of the sleeve and a surface of the port to mitigate disengagement of the port from the coupling device.

In some embodiments, the coupling device further includes a coating configured to provide one of a leak-proof seal or to mitigate tissue ingrowth.

Also disclosed is a method of securing a port to a catheter including, urging a stem of a coupling device into a lumen of the catheter, urging a port stem into a lumen of the coupling device, inverting a sleeve of the coupling device from an unlocked configuration, where a first surface is faced radially inward and a second surface is faced radially outward, to a locked configuration, where the first surface is faced outward and the second surface is faced inward, and engaging the second surface of the sleeve with a portion of the port.

In some embodiments, the sleeve is formed of an elastic material selected from a group consisting of a plastic, polymer, elastomer, silicone rubber, synthetic rubber, natural rubber, latex, or composite.

In some embodiments, the sleeve in the locked configuration defines a recess, a diameter of the recess is the same as or less than a diameter of the portion of the port.

In some embodiments, an inner profile of the recess mirrors and outer profile of the portion of the port.

In some embodiments, the method further includes engaging a ridge, disposed on the second surface of the sleeve, with a suture groove, disposed on the portion of the port, when the sleeve is in the locked configuration.

In some embodiments, the first surface of the sleeve forms a continuous outer profile with a body of the port when the sleeve is in the locked configuration.

In some embodiments, an outer diameter of the stem of the coupling device is the same or larger than an inner diameter of the lumen of the catheter.

In some embodiments, an outer diameter of the port stem is the same or slightly larger than an inner diameter of the lumen of the coupling device to engage the coupling device in a friction fit.

In some embodiments, the method further includes creating an air-tight seal between the second surface of the sleeve and the portion of the port.

In some embodiments, urging the port proximally creates a vacuum within the recess mitigating disengagement between the port and the coupling device.

In some embodiments, the method further includes creating a seal between the second surface and the portion of the port, the seal created by a coating disposed on the second surface, the seal mitigating one of leakage or tissue ingrowth.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows an exploded, perspective view of a port-catheter assembly including a port, a catheter and a coupling device, in accordance with embodiments disclosed herein.

FIG. 2A shows a perspective view of a port, in accordance with embodiments disclosed herein.

FIG. 2B shows a side view of a port, in accordance with embodiments disclosed herein.

FIG. 3A shows a cross-section view of a coupling device in a first, unlocked configuration, in accordance with embodiments disclosed herein.

FIG. 3B shows a cross-section view of a coupling device in a second, locked configuration, in accordance with embodiments disclosed herein.

FIG. 3C shows a perspective view of a coupling device in a first, unlocked configuration, in accordance with embodiments disclosed herein.

FIG. 3D shows a perspective view of a coupling device in a second, locked configuration, in accordance with embodiments disclosed herein.

FIG. 4A shows a horizontal cross-section view of a port-catheter assembly including a port, a catheter and a coupling device, in accordance with embodiments disclosed herein.

FIG. 4B shows a vertical cross-section view of a coupling device and catheter assembly, in accordance with embodiments disclosed herein.

FIG. 4C shows a plan view of a port-catheter assembly including a port, a catheter and a coupling device, in accordance with embodiments disclosed herein.

FIGS. 5A-5F show various steps in an exemplary method of use for a port, a coupling device and a catheter, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

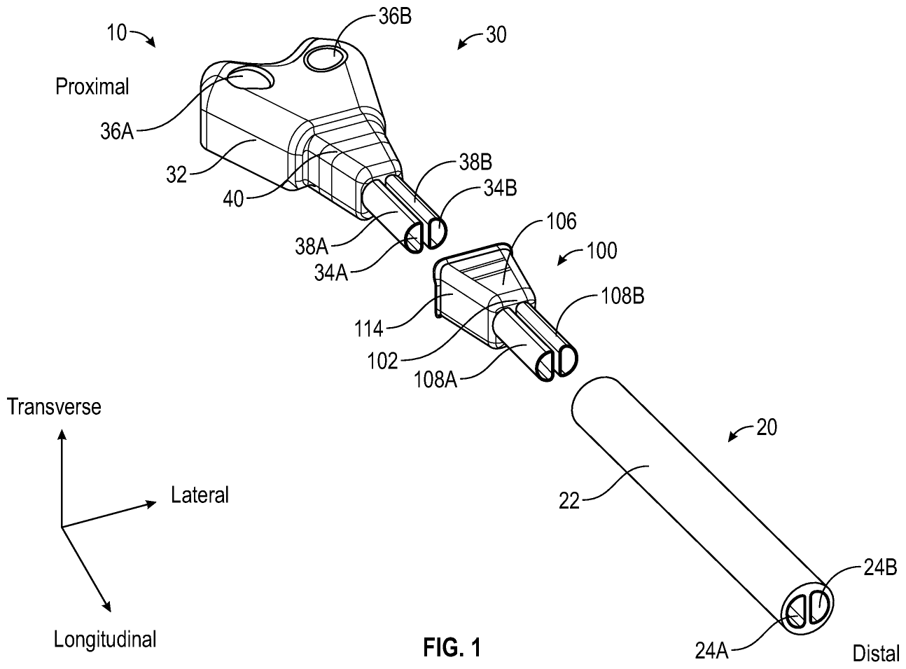

To assist in the description of embodiments described herein, as shown in FIG. 1, a longitudinal axis extends substantially parallel to an axial length of the catheter. A lateral axis extends normal to the longitudinal axis, and a transverse axis extends normal to both the longitudinal and lateral axes. A horizontal plane is defined by the longitudinal and lateral axes. A vertical plane extends normal to the horizontal plane.

Unless defined otherwise, all technical and scientific terms used herein have the same meaning as commonly understood by those of ordinary skill in the art.

FIG. 1 shows an exploded view of a port-catheter assembly 10, generally including a catheter 20, a port 30, and a compliant coupling device or “cathlock” 100 having an invertible, or “bi-stable,” sleeve 106. In an embodiment, the catheter 20 can include a body 22 defining a substantially cylindrical or tubular shape, and can define one or more lumen 24. As shown, the catheter 20 is a dual-lumen catheter including a first lumen 24A and a second lumen 24B. However, it will be appreciated that catheters having greater or fewer lumen are also contemplated to fall within the scope of the present invention.

The port 30 can include a port body 32 defining one or more conduits 34 each configured to communicate with one or more lumen 24 of the catheter 20. For example, a first conduit 34A can communicate with a first lumen 24A, and a second conduit 34B can communicate with a second lumen 24B. The port body 32 can define a flat lower surface to provide stability when placed subcutaneously within a tissue pocket. The port body 32 can further include a rounded upper surface, a rounded upper outer profile shape, or can include rounded edges to facilitate placement within a tissue pocket, mitigating abrasions or trauma to the surrounding tissues improving patient comfort, and mitigating the formation of scar tissue.

The port 30 can further include one or more receiving cups 36 disposed at a proximal end of the port 30. The receiving cup 36 can substantially define a funnel shape, configured to direct a needle inserted therein, into the port conduit 34. As shown, the port 30 includes a first receiving cup 36A communicating with the first conduit 34A, and a second receiving cup 36B communicating with the second conduit 34A. In an embodiment, one or both of the receiving cup 36 and the conduit 34 can further include a needle penetrable septum or valve element configured to control a fluid flow therethrough.

The port 30 can further include a port stem 38, extending from a distal end of the body 32 and configured to engage the coupling device 100 in a friction fit, interference fit, or the like, as described in more detail herein. In an embodiment, the port stem 38 can communicate with the conduit 34 of the port 30. The port 30 can include a first port stem 38A and a second port stem 38B. However, it will be appreciated that the port 30 can have greater or fewer port stems 38. In an embodiment, the port stem 38 can be formed of a resilient or rigid material such as a metal, alloy, plastic, polymer, composite material, or the like. In an embodiment, the port stem 38 can include a ridge or barb 112, extending annularly about the outer surface of the port stem 38 and configured to facilitate engagement of the port stem 38 with one of the coupling device 100 or the catheter 20.

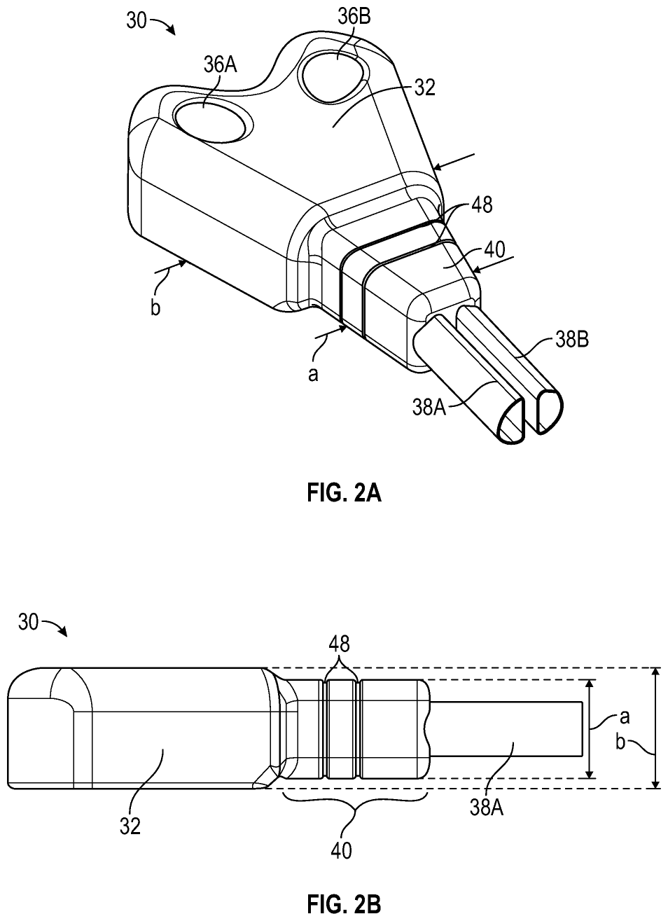

In an embodiment, the port body 32 can include a sleeve bearing portion 40 disposed at a distal end of the port body 32. The sleeve bearing portion 40 can be configured to receive a sleeve portion (“sleeve”) 106 of the coupling device 100, as described in more detail herein. As shown in FIGS. 2A-2B, the sleeve bearing portion 40 can define a thinner lateral or transverse dimension (“a”) than the outer dimensions of the port body 32 (“b”). The difference between the first dimension (“a”) and the second dimension (“b”) being substantially the same as the thickness of the sleeve 106. As such, with the sleeve 106 engaging the sleeve bearing portion 40, i.e. the coupling device 100 is in the locked configuration, the outer profile of the port body 32 and the outer profile of the sleeve 106 form a continuous outer surface, dimension (“b”). Advantageously, the continuous outer surface of the sleeve 106 and port body 32 assembly can mitigate trauma to the tissue pocket, improving patient comfort, and mitigate tissue ingrowth when disposed subcutaneously. In an embodiment, the port body 32 can include one or more suture grooves 48, extending annularly about a portion of the port body 32. The suture groove 48 can be configured to receive a suture therein to secure the port 30 within the tissue pocket.

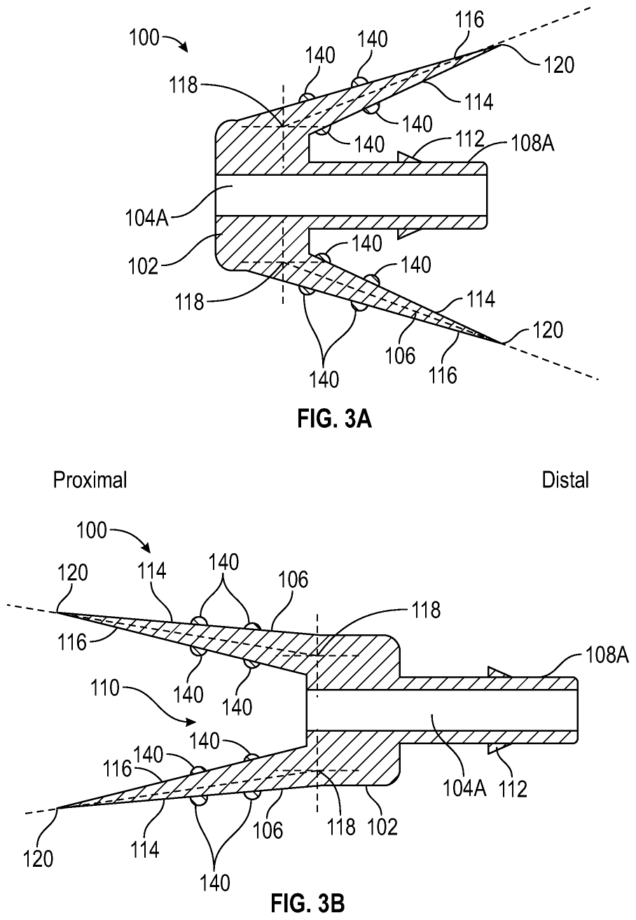

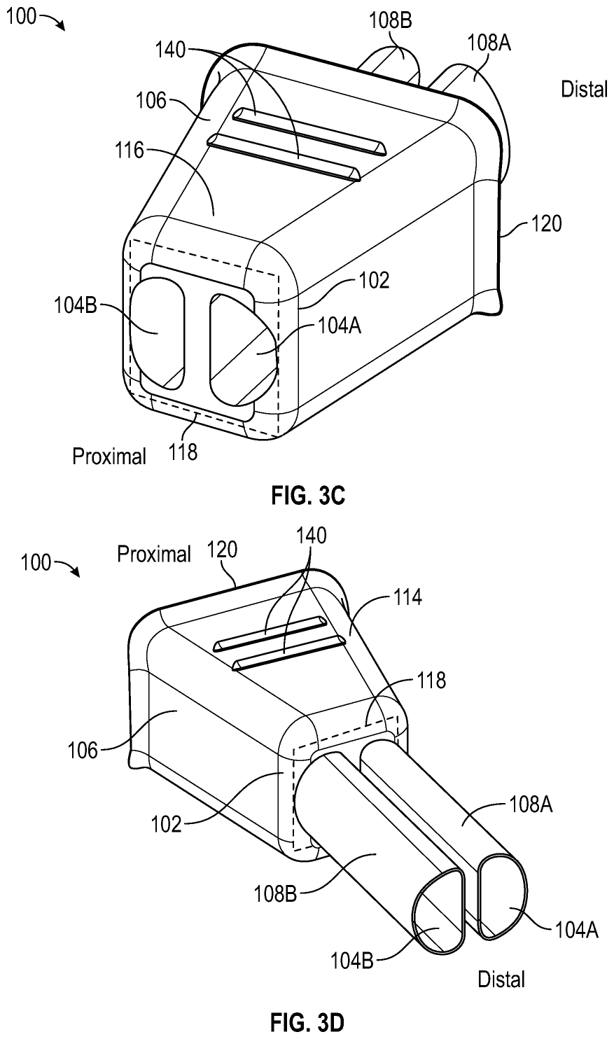

FIGS. 3A-3D show further details of the bi-stable, invertible coupling device 100. FIG. 3A shows a cross-section view of the coupling device 100 in the first, “unlocked” configuration. FIG. 3B shows a cross-section view of the coupling device 100 in the second, “locked” configuration. FIG. 3C shows a proximal end, perspective view of the coupling device 100 in a first, “unlocked” configuration. FIG. 3D shows a distal end, perspective view of the coupling device 100 in second, “locked” configuration.

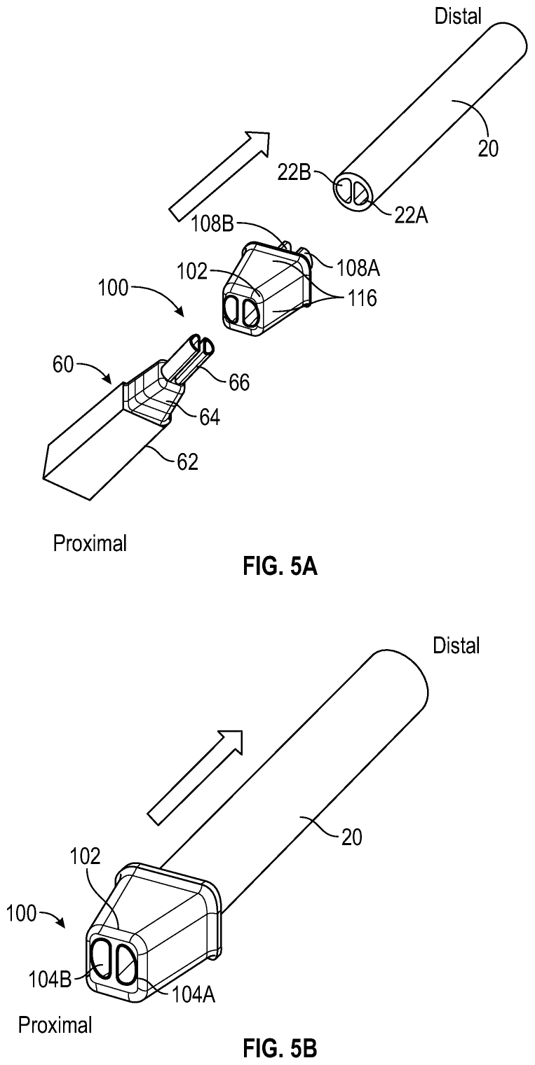

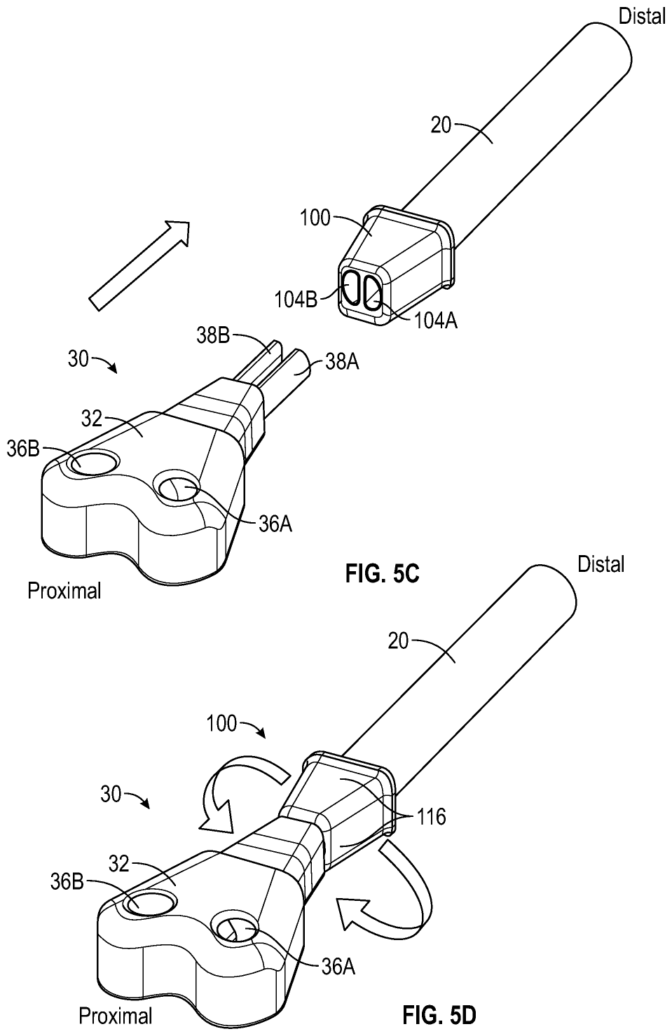

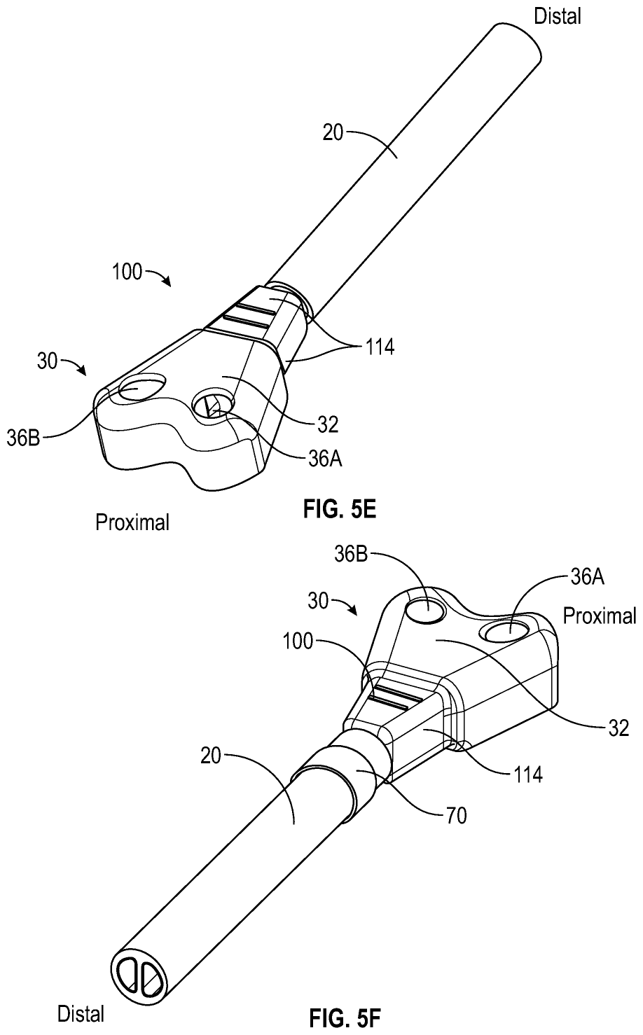

The coupling device or “cathlock” 100 can include a stem receiving portion, or “body” 102 configured to receive the stem 38 of the port 30. The stem receiving portion 102 can further include one or more stems 108 extending from a distal end thereof and configured to engage a lumen of the catheter 20. For example, the cathlock 100 can include a first cathlock stem 108A configured to engage a first lumen 24A, and a second cathlock stem 108B configured to engage a second lumen 24B of the catheter 20. In an embodiment, the stem 108 can be formed of a resilient or rigid material, relative to the material of the catheter 20, such as a metal, alloy, plastic, polymer, composite material, or the like. In an embodiment, the stem 108 can include a ridge or barb 112, extending annularly about the outer surface of the stem 108 and configured to facilitate engagement of the stem 108 with the catheter lumen 24 and secure the catheter 20 to the cathlock 100. The cathlock 100 can further include a lumen 104 extending from through a portion of the body 102 and through the stem 108. The lumen 104 can provide fluid communication between the port stem 38 and the lumen 24 of the catheter 20. In an embodiment, as shown in FIG. 5F, a collar 70 can be slidably engaged with an outer surface of the catheter 20. The collar 70 can be slid proximally, over a portion of the catheter body 20 to compress the catheter body 22 onto the cathlock stem 108, securing the catheter 20 thereto and mitigating leaks.

In an embodiment, the stem receiving portion 102 can include a flexible, invertible sleeve 106 coupled to an outer surface of the stem receiving portion 102 and extending therefrom. The invertible sleeve 106 can be transitionable between a first, unlocked configuration (FIGS. 3A, 3C) and a second, locked configuration (FIGS. 3B, 3D). In the unlocked configuration, the sleeve 106 can extend distally from the body 102. The sleeve 106 can extend about a longitudinal axis. For example, a rim portion 120 of the sleeve 106 can encircle one or both of the body 102 and a portion of the cathlock stem 108. The sleeve 106 can include a first surface 114 and a second surface 116, disposed opposite the first surface 114. In the unlocked configuration, the first surface 114 can face radially inward relative to a central longitudinal axis, and a second surface 116 can face radially outward relative to a central longitudinal axis.

In the locked configuration, the sleeve 106 can extend proximally from the body 102. Worded differently, the sleeve 106 can extend away from the distal end of the stem receiving portion. The sleeve 106 can extend about a longitudinal axis and define a recess 110 configured to receive a portion of the port body 32, e.g. the sleeve bearing portion 40, therein. In an embodiment, the sleeve 106 can substantially define a truncated pyramid shape, with a proximal lateral cross-sectional area being larger than a distal lateral cross-sectional area, i.e. the apex of the truncated pyramid being disposed distally. However, it will be appreciated that the sleeve 106 can also define other three-dimensional shapes without limitation. In the locked configuration, the first surface 114 can face radially outward relative to a central longitudinal axis, and a second surface 116 can face radially inward relative to a central longitudinal axis.

In an embodiment, one or more of the coupling device 100, the stem receiving portion 102, the sleeve 106, or portions thereof, can be formed of a flexible or elastic material such as a plastic, polymer, elastomer, silicone rubber, synthetic rubber, natural rubber, latex, composite, or the like. In an embodiment, the sleeve 106 can be inverted between the first, “unlocked” configuration (FIGS. 3A, 3C) and the second, “locked” configuration (FIGS. 3B, 3D). For example, from the second “locked” stable configuration, a user can fold the sleeve 106, about a hinge axis or fulcrum 118, distally over the cathlock stems 108A, 108B to the first “unlocked” configuration. Similarly, from the first “unlocked” stable configuration, a user can fold the sleeve 106 about the hinge axis or fulcrum 118 proximally to the second, “locked” configuration, to form the recess 110. As such, the coupling device 100 can be bi-stable in both the first “unlocked” configuration as well as the second “locked” configuration. The flexible characteristics of the material of the body 102 allow the body 102 to stretch and to transition between the first, “unlocked” configuration (FIG. 3A) and the second, “locked” configuration (FIG. 3B).

In an embodiment, the coupling device 100 can include one or more ridges 140 extending from one or both of the first surface 114 of the sleeve 106 and the second surface 116 of the sleeve 106. In an embodiment, the ridges 140 disposed on the first surface 114 can align with the suture grooves 48 disposed on the sleeve bearing portion 40 of the port 30, when the port 30 is engaged with the cathlock 100 in the locked configuration. Advantageously, the ridges 140 on the first surface 114 can indicate the location of the sutures, disposed within the suture grooves 48 below the sleeve 106. In an embodiment, a ridge 140 disposed on the second surface 116 of the sleeve 106 can engage a suture groove 48 disposed on the sleeve bearing portion 40. Advantageously, the ridge 140 engaging the suture groove 48 can mitigate longitudinal movement of the coupling device 100 relative to the port 30. In an embodiment, the ridges 140 can define a semi-circular cross-sectional profile. In an embodiment, the ridges 140 can define an angled, or “barbed” profile.

In an embodiment, sutures can encircle both the port 30 and the sleeve 106 in the locked configuration and can be disposed between the ridges 140. As such, the sutures can secure both the port 30 and the coupling device 100 in place within the tissue pocket. The ridges 140 can prevent the sutures from sliding proximally or distally and coming loose.

In an embodiment, the coupling device 100 can include embossed or etched symbols, alphanumeric symbols, different colors, combinations thereof, or the like, configured to indicate either a locked or unlocked configuration. For example, the sleeve 106 can include a locked padlock symbol disposed on the first surface 114 of the sleeve 106 to indicate a locked configuration. Similarly, the coupling device 100 can include an unlocked padlock symbol disposed on the second surface 116 of the sleeve 106. These and similar indicators are contemplated to fall within the scope of the present invention.

In an embodiment, one or more surfaces of the coupling device 100 can include one or more coatings. The coating(s) can be configured to provide a seal between the surface of the coupling device 100 and a surface of one of the catheter 20 or the port 30. In an embodiment, the coating can be impregnated with a drug, or similar active ingredient. In an embodiment, the coating can be configured to provide a leak-proof seal between the coupling device 100 and one of the catheter 20 or the port 30. In an embodiment, the coating can be configured to provide mitigate ingrowth between the coupling device 100 and one of the catheter 20 or the port 30. For example, an outer surface of the stem 108 can include a coating configured to provide a leak-proof seal between the stem 108 and an inner surface of the catheter lumen 24. For example, a second surface 116 of the sleeve 106 can include a coating configured to provide a leak-proof seal and mitigate tissue ingrowth between sleeve 106 and the sleeve bearing portion 40 of the port 30. In an embodiment, the coating can include an adhesive to adhere the one or more surfaces of the coupling device 100 to a surface of one of the catheter 20 or the port 30. These and other combinations of coatings, sealants, drug-impregnated coatings, combinations thereof, or the like, are also contemplated to fall within the scope of the present invention.

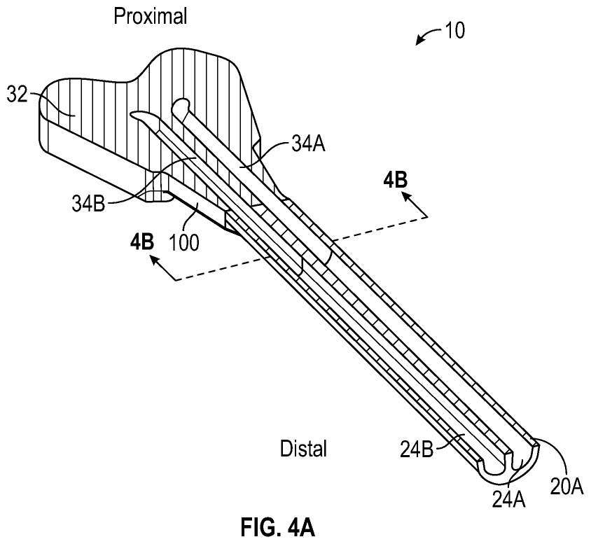

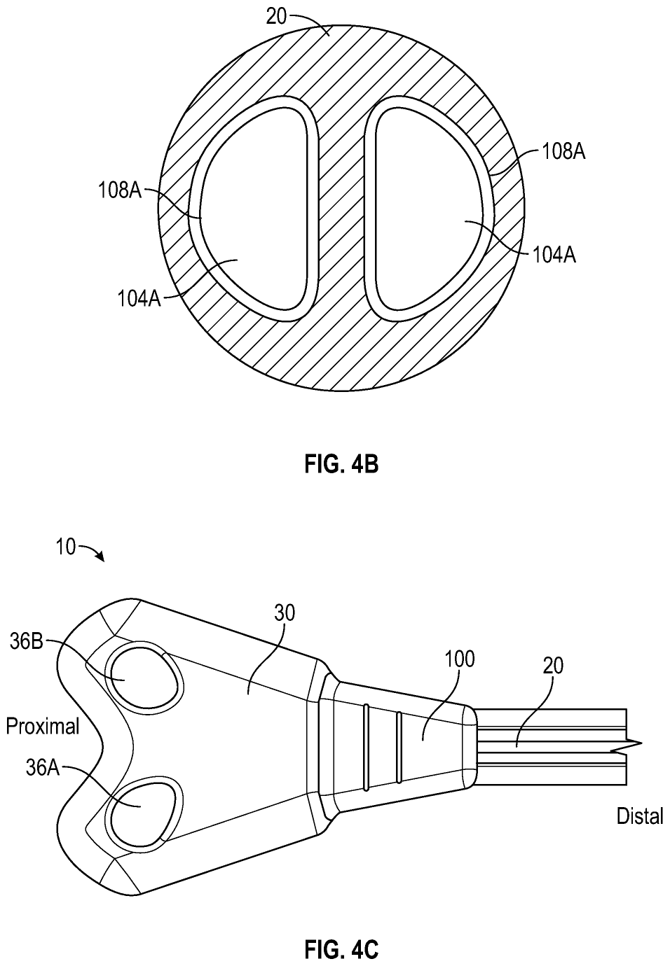

FIGS. 4A-4C show further details of the port-catheter assembly 10. FIG. 4A shows a horizontal cross-sectional view of the port-catheter assembly 10 including the port 30, the coupling device 100, and the catheter 20. FIG. 4B shows a cross-section view of the cathlock stems 108A, 108B disposed within the lumen 24 of the catheter 20. FIG. 4C shows a plan view of the port-catheter assembly 10, including the port 30, coupling device 100, and the catheter 20.

In an exemplary method of use, as shown in FIGS. 5A-5F, a port-catheter assembly 10 is provided, including a catheter 20, a port 30, and a compliant coupling device or “cathlock” 100, as described herein. In an embodiment, a distal portion of the catheter 20 can be disposed at a target location within the vasculature of the patient. A proximal portion of the catheter 20 can be disposed in a tissue pocket and optionally trimmed to a suitable length. In an embodiment, the port 30 can be secured within the tissue pocket and the proximal end of the catheter 20 can be coupled thereto, within the tissue pocket. In an embodiment, the port 30 can be coupled to the proximal end of the catheter 20 and then placed within the tissue pocket.

In an embodiment, as shown in FIGS. 5A-5B, the coupling device 100 can be provided in the unlocked configuration and can engage a proximal end of the catheter 20. The stem 108 can be urged into the catheter lumen 24 and can engage therewith in a friction fit, interference fit, or press fit engagement. In an embodiment, an outer diameter of the stem 108 can be the same or slightly larger than an inner diameter of the catheter lumen 24. In an embodiment, the stem 108 can be formed of a resilient or rigid material, relative to the catheter 20. In an embodiment, the catheter 20 can be formed of a compliant, flexible, or elastic material, for example a polymer, plastic, elastomer, rubber, latex, or the like. As such, the catheter lumen 24 can be stretched over the stem 108 and secured thereto. With the coupling device 100 engaged with the catheter (FIG. 5B), the lumen 104 of the coupling device 100 can be in fluid communication with the lumen 24 of the catheter 20.

In an embodiment, a tool 60 can engage with the coupling device 100 to facilitate securing the coupling device 100 with the catheter 20. For example, the tool 60 can include a handle 62 and a proximal sleeve bearing portion 64 shaped similarly to the sleeve bearing portion 40 of the port 30. In an embodiment, the tool 60 can further include one or more protrusions 66 extending from a distal end thereof and configured to engage a lumen 104 of the coupling device 100. In an embodiment, the sleeve 106 can transition to the locked configuration to lock the coupling device 100 to the sleeve bearing portion 64 of the tool 60. Once the coupling device 100 is engaged with the catheter 20, the sleeve 106 can be transitioned to the unlocked configuration to disengage the tool 60. In an embodiment, the tool protrusions 66 can engage the lumen 104 of the coupling device 100 to engage the coupling device 100 with the tool 60. In an embodiment, the coupling device 100 can be provided preassembled with the catheter 20 in the unlocked configuration.

As shown in FIGS. 5C-5D, with the coupling device 100 engaged with the proximal end of the catheter 20, the port 30 can then be engaged with the coupling device 100. The port stem 38 can be urged into the lumen 104 of the coupling device 100. In an embodiment, the outer diameter of the port stem 38 can be the same or slightly larger than an inner diameter of the lumen 104. As such, the port stem 38 can engage the stem receiving portion 102 of the coupling device 100 in a friction fit, interference fit, or press fit engagement.

As shown in FIGS. 5D-5E, with the port stem 38 engaged with the lumen 104, the user can then invert the sleeve 106 from the first, unlocked configuration (FIG. 5D) to the second, locked configuration (FIG. 5E), as described herein. Folding the sleeve 106 over, about the fulcrum 118, to the locked configuration engages a second surface 116 of the sleeve 106 with a sleeve bearing portion 40 of the port 30. The sleeve 106 in the locked configuration defines a recess 110 configured to receive the sleeve bearing portion 40 therein. An inner diameter of the recess 110 can be less than an outer diameter of the sleeve bearing portion 40. As such, the sleeve 106 can fit tightly over the sleeve bearing portion 40 gripping and retaining the sleeve bearing portion 40 therein. In an embodiment, an inner profile of the recess 110 can mirror an outer profile of the sleeve bearing portion 40 of the port 30. In an embodiment, the inner profile of the recess 110 can substantially define a truncated pyramid shape. However, it will be appreciated that other profile shapes are also contemplated.

In an embodiment, the sleeve 106 can create an air tight seal between the second surface 116 and the port 30. Advantageously, any proximal force urging the port 30 away from the coupling device 100 can create a vacuum force within the recess 110 of the sleeve 106, mitigating disengagement of the port 30 from the coupling device 100. In an embodiment, a ridge 140 disposed on the second surface 116 of the sleeve 106 can engage a suture groove 48 disposed on the sleeve bearing portion 40 of the port 30, when the sleeve 106 is in the locked configuration. Advantageously, the engagement between the ridge 140 and the suture groove 48 can mitigate disengagement of the port 30 from the coupling device 100.

In an embodiment, when the coupling device 100 is engaged with the port 30 and the sleeve 106 is in the locked configuration, the outer surface of the sleeve 106 can form a continuous outer profile with the outer surface of the port 30. Advantageously, the continuous outer profile can mitigate the formation of tissue ingrowth when the port-catheter assembly 10 is disposed subcutaneously.

In an embodiment, as shown in FIG. 5F, a collar 70 can engage an outer surface of the catheter 20 and compress the catheter body 22 on to the stem 108 of the coupling device 100 to further secure the catheter 20 to the coupling device 100, mitigating leaks. In an embodiment, a user can tie sutures about the catheter 20 secure the catheter to the cathlock stem 108.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A coupling device for releasably securing a catheter to a port, comprising:
a stem receiving portion configured to receive a stem of the port, the stem receiving portion comprising a lumen; and
an invertible sleeve coupled to an outside surface of the stem receiving portion, the invertible sleeve comprising:
a first configuration extending toward a distal end of the stem receiving portion; and
a second configuration extending away from the distal end of the stem receiving portion.

2. The coupling device according to claim 1, wherein a rim portion of the invertible sleeve in the first configuration extends distally from the stem receiving portion to surround a portion of the stem receiving portion.

3. The coupling device according to claim 1, wherein the invertible sleeve in the second configuration extends proximally to define a recess configured to receive a portion of the port therein.

4. The coupling device according to claim 3, wherein the portion of the port is a sleeve bearing portion.

5. The coupling device according to claim 4, wherein a diameter of the recess is the same as, or less than, a diameter of the sleeve bearing portion of the port.

6. The coupling device according to claim 4, wherein an inner profile of the recess mirrors and outer profile of the sleeve bearing portion of the port.

7. The coupling device according to claim 3, wherein an inner surface of the recess includes a ridge configured to engage a suture groove disposed on the port, when the invertible sleeve is in the second configuration.

8. The coupling device according to claim 1, wherein an outer surface of the invertible sleeve in the second configuration and an outer surface of a body of the port forms a continuous outer profile.

9. The coupling device according to claim 1, wherein the sleeve is formed of an elastic material selected from a group consisting of a plastic, polymer, elastomer, silicone rubber, synthetic rubber, natural rubber, latex, or composite.

10. The coupling device according to claim 1, further including a stem extending distally from the stem receiving portion and configured to engage a lumen of a catheter.

11. The coupling device according to claim 10, wherein an outer diameter of the stem of the coupling device is the same or larger than an inner diameter of a lumen of a catheter to engage the catheter in a friction fit.

12. The coupling device according to claim 1, wherein an outer diameter of the stem of the port is the same or slightly larger than an inner diameter of the lumen of the stem receiving portion.

13. The coupling device according to claim 1, wherein the sleeve in the second configuration is configured to create an air tight seal between a surface of the sleeve and a surface of the port to mitigate disengagement of the port from the coupling device.

14. The coupling device according to claim 1, further including a coating configured to provide one of a leak-proof seal or to mitigate tissue ingrowth.

15. A method of securing a port to a catheter, comprising:
urging a stem of a coupling device into a lumen of the catheter;
urging a port stem into a lumen of the coupling device;
inverting a sleeve of the coupling device from an unlocked configuration, where a first surface is faced radially inward and a second surface is faced radially outward, to a locked configuration, where the first surface is faced outward and the second surface is faced inward; and
engaging the second surface of the sleeve with a portion of the port.

16. The method according to claim 15, wherein the sleeve is formed of an elastic material selected from a group consisting of a plastic, polymer, elastomer, silicone rubber, synthetic rubber, natural rubber, latex, or composite.

17. The method according to claim 15, wherein the sleeve in the locked configuration defines a recess, a diameter of the recess is the same as or less than a diameter of the portion of the port.

18. The method according to claim 17, wherein an inner profile of the recess mirrors and outer profile of the portion of the port.

19. The method according to claim 15, further including engaging a ridge, disposed on the second surface of the sleeve, with a suture groove, disposed on the portion of the port, when the sleeve is in the locked configuration.

20. The method according to claim 15, wherein the first surface of the sleeve forms a continuous outer profile with a body of the port when the sleeve is in the locked configuration.

21. The method according to claim 15, wherein an outer diameter of the stem of the coupling device is the same or larger than an inner diameter of the lumen of the catheter.

22. The method according to claim 15, wherein an outer diameter of the port stem is the same or slightly larger than an inner diameter of the lumen of the coupling device to engage the coupling device in a friction fit.

23. The method according to claim 15, further including creating an air-tight seal between the second surface of the sleeve and the portion of the port.

24. The method according to claim 23, wherein urging the port proximally creates a vacuum within the recess mitigating disengagement between the port and the coupling device.

25. The method according to claim 24, further including creating a seal between the second surface and the portion of the port, the seal created by a coating disposed on the second surface, the seal mitigating one of leakage or tissue ingrowth.
